(19) United States
(12) Patent Application Publication
(10) Pub. No.: US 2020/0258599 A1
(43) Pub. Date: Aug. 13, 2020

(54) METHODS AND SYSTEMS FOR PREDICTING CLINICAL TRIAL CRITERIA USING MACHINE LEARNING TECHNIQUES

(71) Applicant: International Business Machines Corporation, Armonk, NY (US)

(72) Inventors: Adam CLARK, MANTORVILLE, MN (US); Eric W. WILL, ROCHESTER, MN (US); David RANUM, Decorah, IA (US); Luis Alexandro GARCIA, AUSTIN, TX (US); Kimberly Diane KENNA, Cary, NC (US)

(21) Appl. No.: 16/273,475

(22) Filed: Feb. 12, 2019

(51) Int. Cl. G16H 10/20; G06K 9/62; G06N 20/20

(52) U.S. Cl. CPC G16H 10/20; G06N 20/20; G06K 9/6256

(57) ABSTRACT

A method and apparatus for identifying contextual information related to clinical trial criteria using machine learning techniques is disclosed. An example method generally includes training a machine learning (ML) model to identify an intended respondent for a criterion. A system receives a plurality of criteria associated with a first clinical trial and determines a respective intended respondent for each of the plurality of criteria based on analyzing the plurality of criteria using the ML model. The system associates each of the plurality of criteria with an indication of the corresponding intended respondent.

### BACKGROUND

The present invention relates to using machine learning techniques to deliver clinical trial recommendations, and more specifically, to identifying who should evaluate clinical trial eligibility criteria for a patient and when clinical trial eligibility criteria should be evaluated in matching patients to clinical trials.

Clinical trials in medicine are research studies that are used to test and evaluate various medical treatments, drugs, or devices under development. Typically, clinical trials are defined as a treatment, drug, or device being developed, eligibility criteria (or inclusion criteria) defining the characteristics of patients who may be eligible to participate in a specified trial, and disqualifying criteria defining the characteristics of patients who are not eligible for participation in the trial. For example, the eligibility criteria may include the medical condition that the subject of the clinical trial is addressing, a stage of medical treatment that patients should be at, what previous treatments a patient may have received prior to entering the clinical trial, and the like. The disqualifying criteria defining the characteristics of patients who are not eligible to participate in a specified trial may include, for example, a stage of a disease beyond which a patient would be ineligible for inclusion in the trial, previous treatments that disqualify a patient from participating in the trial, and the like. While clinical trial eligibility and disqualifying criteria may be written according to a standard format, the eligibility and disqualifying criteria and other relevant information about clinical trials may not be written in a clear and concise manner.

At any given time, a patient may potentially be eligible for participation in a variety of clinical trials. Typically, to determine what clinical trial(s) a patient may be eligible for participation in, the patient's doctors and/or other clinical staff may review the patient's medical records and the eligibility and disqualifying criteria for a number of clinical trials to identify trials that may be of interest to the patient. However, the process of identifying trials that are potentially of interest for the patient may be a time consuming, manual process that requires doctors or other clinical staff to compare potentially voluminous patient records with at least the eligibility and disqualifying criteria for each clinical trial. Further, due to the number and wide variety of clinical trials that may be active at any time, manual searches for trials of interest may miss potentially relevant trials for a given patient. In some cases, manual analysis of potential clinical trials to enroll a patient in may rely on institutional procedures that prioritize clinical trials being run in certain institutions over potentially relevant clinical trials run in other institutions, which may result in potentially relevant clinical trials for a patient being overlooked or otherwise omitted from consideration.

Automated methods for analyzing patient records and clinical trial definitions may not be able to accurately match patients with the clinical trials that patients may be eligible to participate in for various reasons. For example, automated methods may not be able to accurately parse the intent of statements in a clinical trial definition. In another example, automated methods may not be able to understand the implications of a patient's records with respect to the ability to successfully complete a clinical trial. In still further examples, automated methods may be unable to determine or identify temporal relationships associated with eligibility or disqualifying criteria for a clinical trial.

### SUMMARY

One embodiment of the present disclosure provides a method for identifying contextual information for clinical trial enrollment criteria. The method generally includes training a machine learning (ML) model to identify an intended respondent for a criterion. A system receives a plurality of criteria associated with a first clinical trial and determines a respective intended respondent for each of the plurality of criteria based on analyzing the plurality of criteria using the ML model. The system associates each of the plurality of criteria with an indication of the corresponding intended respondent.

Another embodiment of the present disclosure provides a system having a processor and a memory. The memory generally has instructions stored thereon which, when executed by the processor, performs an operation for identifying contextual information for clinical trial enrollment criteria. The operation generally includes training a machine learning (ML) model to identify an intended respondent for a criterion. A system receives a plurality of criteria associated with a first clinical trial and determines a respective intended respondent for each of the plurality of criteria based on analyzing the plurality of criteria using the ML model. The system associates each of the plurality of criteria with an indication of the corresponding intended respondent.

Still another embodiment of the present disclosure provides a computer-readable medium having instructions stored thereon which, when executed by a processor, performs an operation for identifying contextual information for clinical trial enrollment criteria. The operation generally includes training a machine learning (ML) model to identify an intended respondent for a criterion. A system receives a plurality of criteria associated with a first clinical trial and determines a respective intended respondent for each of the plurality of criteria based on analyzing the plurality of criteria using the ML model. The system associates each of the plurality of criteria with an indication of the corresponding intended respondent.

### BRIEF DESCRIPTION OF THE SEVERAL VIEWS OF THE DRAWINGS

FIG. 1 illustrates an example networked environment in which machine learning models are used to predict intended respondents and timing for evaluating clinical trial eligibility for a patient, according to one embodiment.

FIG. 2 illustrates example operations for training a machine learning model for predicting intended respondents and timing for evaluating clinical trial eligibility for a patient, according to one embodiment.

FIG. 3 illustrates example operations for predicting intended respondents and timing for evaluating clinical trial eligibility for a patient using a trained machine learning model, according to one embodiment.

FIG. 4 illustrates an example system in which aspects of the present disclosure may be performed.

### DETAILED DESCRIPTION

Embodiments presented herein describe techniques for identifying intended respondents and timing of a response in evaluating whether a patient is eligible for a clinical trial using machine learning techniques. As discussed, clinical trials are generally defined by eligibility criteria, which indicates which patients may be enrolled into a trial, and disqualifying criteria, which are conditions, previous treatments, etc. that prevent patients from being enrolled into the trial. However, clinical trial specifications may not be written clearly and may lack detail, leading to ambiguities in identifying what the eligibility and disqualifying criteria are and thus difficulty in understanding whether a patient is eligible to participate in a specific clinical trial. Additionally, different criteria may be best answered by different clinicians, and at different times. However, because of clinical trial specifications generally do not specify who should evaluate a criterion and when such a criterion should be evaluated, clinical trial management systems may list the eligibility and disqualifying criteria for a clinical trial without providing any additional information about the eligibility and disqualifying criteria. The listing of eligibility and disqualifying criteria for a clinical trial without additional contextual information may, for example, cause some patients to be improperly included or excluded from a clinical trial. Still further, eligibility and disqualifying criteria may be treated as binary options (i.e., answerable either in the affirmative or the negative), when some criteria may include a more granular set of possible answers.

As discussed herein, labeled data about the eligibility and disqualifying criteria for a plurality of clinical trials may be used to train a machine learning model to analyze criteria specified for a clinical trial and recommend who should provide information about a specific criterion and when such information should be provided. By adding information about who should provide information related to specific eligibility or disqualifying criteria and when such information may be provided, techniques described herein may provide additional information to a user (e.g., a clinician treating a patient) to aid in accurately and efficiently determining whether a patient is eligible for participation in a trial. This additional information provided to a user may reduce the likelihood of false positives (e.g., recommending to a patient a trial that the patient is not eligible to participate in) and false negatives (e.g., failing to recommend to a patient a trial that the patient is actually eligible to participate in). Embodiments presented herein may thus improve clinical trial management systems by augmenting information included in a definition of a clinical trial with information that may aid users in analyzing whether a patient is eligible to participate in such a trial.

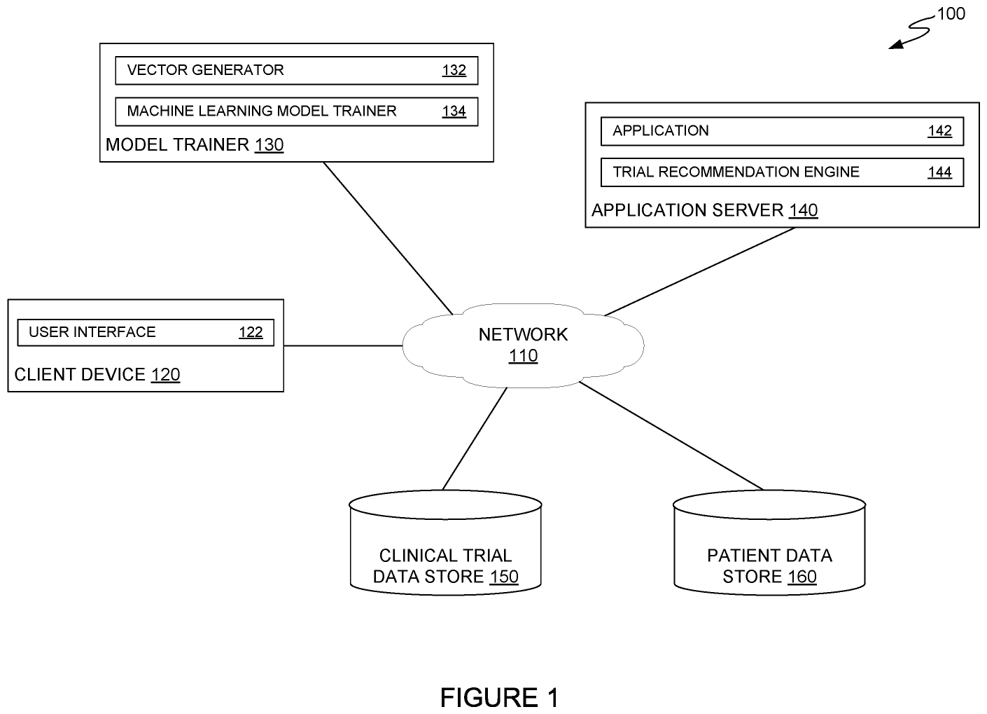

FIG. 1 illustrates an example networked computing environment in which machine learning models are used to identify who should provide information about clinical trial eligibility and disqualifying criteria and when such information should be provided, according to an embodiment of the present disclosure. As illustrated, computing environment 100 includes a client device 120, a model trainer 130, an application server 140, a clinical trial data store 150, and a patient data store 160, connected via network 110.

Client device 120 generally is representative of a computing device on which a user can define and/or manage the training of predictive models used by trial recommendation engine 144 to recommend potentially relevant clinical trials for a patent and access application 142 on application server 140 to obtain a set of potentially relevant clinical trials for a patient and analyze eligibility and disqualifying criteria for one or more trials in the set of potentially relevant clinical trials. Client device 120 may be, for example, a laptop computer, a desktop computer, a thin client, a tablet computer, a mobile computing device, and the like. As illustrated, client device 120 includes a user interface 122. User interface 122 allows a user of client device 120 to define a training data set for use in training machine learning models for identifying eligibility and disqualifying criteria in a clinical trial, classes of persons qualified to provide information about each of the eligibility and disqualifying criteria, and timing information identifying when information should be provided about each of the eligibility and disqualifying criteria. User interface 122 additionally allows a user of client device 120 to initiate a search for recommended clinical trials that may be of interest to a patient by providing, to application 142, the patient's medical records in a request for one or more potentially relevant clinical trials to present to a patient. For each of the one or more potentially relevant clinical trials returned by application 142 on application server 140, the eligibility and disqualifying criteria as well as information generated by a machine learning model identifying who should provide information about clinical trial eligibility and disqualifying criteria and when such information should be provided may be displayed to a user for further analysis.

Model trainer 130 generally uses information about patients previously enrolled in trials and the criteria defined for those trials to train one or more machine learning models used in recommending clinical trials that are potentially relevant to a particular patient and identifying, for one or more of the recommended clinical trials, who should provide information about clinical trial eligibility and disqualifying criteria and when such information should be provided. As illustrated, model trainer 130 includes a vector generator 132 and a machine learning model trainer 134.

Vector generator 132 is generally configured to generate a training data set for use by machine learning model trainer 134 to train a machine learning model for recommending potentially relevant clinical trials to a user based on patient medical history. To generate the training data set, vector generator 132 can obtain information about previously completed from clinical trial data store 150 and patient medical history data from patient data store 160. The information obtained from clinical trial data store 150 may include, for example, a roster of patients enrolled in a specific clinical trial and a definition of that clinical trial. The definition of the clinical trial may include eligibility and disqualifying criteria, patient requirements for participation in the trial, a trial enrollment deadline, and other information defining the clinical trial. The roster of patients may include information identifying each patient that vector generator 132 can use to obtain patient medical records from patient data store 160.

To generate the training data to be used by machine learning model trainer 134, vector generator can generate a first set of training data comprising feature data and label data used to train a machine learning model and a second set of unlabeled feature data that can be used to test the generated machine learning model. For example, in an embodiment where machine learning techniques are used to recommend relevant trials for a patient, the first set of data may comprise a plurality of vectors, where the features in each vector include information from patient medical records, and the labels in each vector include the characteristics of a given clinical trial (e.g., eligibility criteria and disqualifying criteria defined for a clinical trial). The second set of data may comprise an unlabeled set of patient medical records associated with patients who have been accepted into a clinical trial.

In some embodiments, the features in the first set of data may further include additional data that may be used to further refine recommendations of trials that may be relevant to a patient. This additional data may include, for example, information about a current stage of treatment that a patient is in, the specialty of the patient's clinicians, the institution that is treating the patient, and patient completion success for other trials that the patient may have participated in. These additional features may be used to further refine the recommendations delivered by trial recommendation engine 144 to deliver more relevant recommendations to the patient's doctors. For example, the use of information about a stage of treatment that the patient is in may be used to prioritize recommendations of trials relevant to that particular stage of treatment over trials relevant to earlier or later stages of treatment (e.g., where a patient is in an early stage of a disease, prioritizing clinical trials directed to curative treatments over palliative treatments that are more appropriate for patients with later or terminal stages of the disease). Likewise, using information about the patient's clinicians may further indicate, at least implicitly, relevant information about the patient's conditions, such as disease progression, that may be used to prioritize some clinical trials over others. This information may, for example, prioritize clinical trials being held at particular institutions (e.g., based on a distance metric from the patient's clinicians) based on assumptions that patients are more likely to successfully participate in trials that are more easily accessible to the patient. Finally, information about the patient's previous trial completion success may be used as an input to prioritize trials, for example, with similar or less stringent completion requirements to trials that the patient has previously successfully participated in. It should be noted, however, that these additional data points for refining the recommendation of relevant trials is not exhaustive, and other appropriate data points may be used to train predictive models for delivering recommendations of potentially relevant clinical trials for a given user.

As discussed herein, the vectors generated by vector generator 132 for the first and second training data sets may be generated using a variety of techniques. In some embodiments, the vectors may be generated from a corpus of clinical trials using natural language processing (NLP) techniques such as the Bag of Words Model or Term Frequency and Inverse Document Frequency (TD-IDF) Model. Other NLP techniques, such as the word2vec algorithm or other neural network-based algorithms, may also be used to create vectors for the first and second training data sets. Additionally, key concepts, logical parse, key criterion triggers, and other non-NLP techniques may be used to generate vectors from corpuses of clinical trials. Criterion triggers may include, for example, hypothetical spans, negations, ignorable passages, and other criteria that may be used to identify relevant information in a clinical trial specification to be included in a vector.

Machine learning model trainer 134 generally is configured to obtain the training data generated by vector generator 132 and, using supervised learning techniques, train one or more predictive models for delivering recommendations of potentially relevant clinical trials for a patient.

In some embodiments, machine learning model trainer 134 may train a first machine learning model used to identify an initial set of clinical trials that are likely to be relevant to a patient based on patient medical data and the characteristics of each clinical trial (e.g., eligibility and exclusion criteria). To train the first machine learning model, recommendation engine trainer 134 can utilize supervised learning techniques using the first training data set discussed above. The labeled data in the first training data set may be used to initially train the first machine learning model, and a user may test the initially trained first machine learning model using the unlabeled data in the first training data set to verify that the first machine learning model returns accurate results (e.g., a recommended set of clinical trials including one or more trials that a patient actually enrolled in) and, if needed, further refine the trained machine learning model based on real-life clinical trial enrollment data associated with a given patient in the unlabeled data.

Further, machine learning model trainer 134 may be further configured to train a second machine learning model to generate context information about clinical trial eligibility and disqualifying criteria based on a training data set of labeled data associating each criterion in a clinical trial specification to contextual information identifying, for example, who should provide information related to the criterion and when such information should be provided in order to make a trial eligibility determination for a patient. Machine learning model trainer 134 may use supervised learning techniques to train the second machine learning model using a plurality of labeled trial specifications as training data. The plurality of labeled trial specifications may be stored, for example, in clinical trial data store 150. Each trial specification may include a plurality of criteria, where each criterion is labeled with one or more contextual items. The contextual items may include, for example, information identifying who can provide information related to the criterion, when such information can be provided to make an eligibility determination for a patient, and other information that may indicate the actual meaning of a criterion in the trial specification. In some embodiments, some of the contextual information may be determined using heuristic methods, natural language processing, or other processes that may infer the actual meaning of a criterion from its position in the trial specification. For example, a trial specification including the phrase “Major surgery within 2 weeks prior to registration” in eligibility or disqualifying criteria generally indicates a temporal aspect that may be combined with the contextual information of positioning of the phrase in eligibility or disqualifying criteria to identify who should provide information about the criterion and when such information should be provided. Using natural language processing and/or heuristic techniques, machine learning model trainer 134 can extract the likely true meaning of the criterion and train a machine learning model appropriately.

The labels applied to various criterion in a trial specification may vary in granularity. For example, in some embodiments, a criterion may be labeled with information identifying that a criterion requires patient consent or that the criterion should be answered by a medical professional. In other embodiments, a criterion may be labeled with more granular information, such as information identifying that a criterion should be answered by the patient, the patient's legal guardian, the patient's doctor, the patient's nurses, a trial coordinator, or other personnel who may have knowledge of the information needed to make a determination with respect to the criterion. In still further embodiments, a criterion may be labeled with other information specifying a qualification of a user needed to provide a response to a particular criterion in a trial specification. For example, suppose that a clinical trial specification includes, in eligibility or disqualifying criteria, the criterion: “Uncontrolled, significant intercurrent or recent illness including, but not limited to, ongoing or active infection, in the opinion of the treating investigator.” This phrase explicitly identifies information about who should resolve this criterion. However, suppose that a clinical trial specification includes in eligibility or disqualifying criteria, the criterion: “Uncontrolled, significant intercurrent or recent illness including, but not limited to, ongoing or active infection,” which does not explicitly identify who should resolve this criterion. Using natural language processing techniques and contextual information supplied by words in the criterion like “uncontrolled” when combined with terms like “illness” or “infection,” it may be inferred that a medical professional, rather than a person without a medical background, should provide information to resolve this criteria.

In some embodiments, information identifying when information related to an eligibility or disqualifying criterion should be provided or analyzed may be defined relative to a clinical event. In these cases, eligibility or disqualifying criterion may be evaluated only after a specific event has occurred, such as the performance of a medical scan (e.g., an magnetic resonance imaging (MRI) scan, a computed tomography (CT) scan, etc.) and analysis of the scan by a qualified person (e.g., the patient's treating clinician), the performance of a specified procedure, and the like.

After training the second machine learning model, machine learning model trainer 134 may deploy the model for use by application 142 and/or trial recommendation engine 144, as described in further detail below. Generally, deployment of the model for use by application 142 may be performed when analysis of a clinical trial is to be performed on an on-demand basis (e.g., when a clinician is reviewing a set of recommended clinical trials to identify one or more trials that may benefit the patient). Deployment of the model for use by trial recommendation engine 144 may be performed when analysis of a clinical trial is to be performed as part of generating a recommended set of clinical trials (e.g., to exclude potentially relevant trials returned from execution of the first machine learning model that the patient would not be eligible to participate in). In some embodiments, the second machine learning model may be deployed as an independent application on application server 140 that is configured to aperiodically or periodically scan clinical trial data store 150 to identify trials that have been added to clinical trial data store 150 since the last scan and add contextual information to the newly identified trials.

In some embodiments, recurrent algorithms may be used to train the machine learning models described herein. Using recurrent algorithms, such as a Recurrent Neural Network (RNN), the machine learning models may be configured to return one or more values indicative of a likelihood, for example, that a particular clinical trial is relevant to a patient or that a particular implied criterion is applicable to a given clinical trial. Further, by using RNNs to train the machine learning models described herein, the machine learning models can analyze clinical trial specifications in the scope of continuous sequences, such as sentences or phrases in a clinical trial specification. In some embodiments, classification type algorithms, where the output of a trained machine learning model is an identified category, may also be used to identify types of clinical trials that a user may be eligible for participation in and implied criteria applicable to a given clinical trial.

Application server 140 generally includes an application 142 and a trial recommendation engine 144. Application 142 may be any type of application in which users can request recommendations of potentially relevant clinical trials for a patient by providing patient data (e.g., medical condition information, treatment history, prior clinical trial participation history, and other relevant information) and, in some embodiments, user-defined filters in a search request executed by application 142. Client device 120 may instantiate or initiate a session of application 142 in response to a request for application content (e.g., a list of active clinical trials that are enrolling patients for participation) generated by a user of client device 120. In some embodiments, the instance of a session of application 142 may be instantiated by a user of client device 120 accessing a home page of an application 142 structured as a web application. In other embodiments, user interface 122 may instantiate the instance of application 142 by launching an executable file on client device that includes components that execute locally on client device 120 and use data provided by application 142.

During execution of application 142, a user may request a set of recommended clinical trials for a given patient by providing that patient's medical data to application 142 in conjunction with a search request. In response, application 142 provides the received medical data to trial recommendation engine 144 for analysis. Application 142 may receive a list of potentially relevant clinical trials for the patient from trial recommendation engine, as discussed in further detail below, and display the list of potentially relevant clinical trials in user interface 122 of client device 120. In some embodiments, the list of potentially relevant clinical trials may include a predetermined number of potentially relevant clinical trials for the patient and may be sorted based on the predictive scores associated with each of clinical trial in the list of potentially relevant clinical trials. Application 142 may additionally allow a user of client device 120 to further refine the list of potentially relevant clinical trials using one or more user-defined filters.

In some embodiments, application 142 may additionally be configured to aperiodically (e.g., upon user request) or periodically analyze sets of clinical trial specifications using the second machine learning model to augment clinical trial specifications with contextual information, such as information about who is qualified to make an eligibility or ineligibility decision with respect to trial criteria, when such a decision may be made, and the like. Unlabeled clinical trials (e.g., clinical trials added to clinical trial data store 150 after a previous analysis of clinical trials in clinical trial data store 150) may be analyzed independent of patient data in this manner. Analyzing clinical trials independent of patient data may allow for the addition of contextual information prior to receiving requests for recommended clinical trials for a patient, which may be less resource intensive than integrating criteria classification into a request for recommended clinical trials for a patient.

Trial recommendation engine 144 uses the machine learning model generated by recommendation engine trainer using the first training data set 134 to examine medical records for a given patient and recommend potentially relevant clinical trials for the patient to the patient's clinicians based, at least in part, on the patient's medical history. Techniques for doing so are described in U.S. patent application Ser. No. ______ of Clark et al., filed ______, 2019 and entitled “Intelligent Ranking of Trials for a Patient” (Attorney Docket No. P201805620), the contents of which are herein incorporated by reference.

In some embodiments, trial recommendation engine 144 may further process the set of potentially relevant clinical trials using the second machine learning model and patient data provided as input into the first machine learning model for generating the set of potentially relevant clinical trials. By further processing the set of recommended trials using the second machine learning model, embodiments of the present disclosure can annotate the recommended set of clinical trials with contextual information that will aid the user in determining whether a patient is eligible to participate in a trial. Further, processing the set of potentially relevant clinical trials using the second machine learning model may additionally be used to refine the set of potentially relevant clinical trials by identifying trial criteria that can be evaluated using which information that is already available in patient data store 160. Based on the evaluations of the identified trial criteria and information already available in patient data store 160 for the patient, trial recommendation engine 144 can remove trials from the set of potentially relevant clinical trials that the patient is ineligible for.

In some embodiments, application 142 may display information about a clinical trial to a user via user interface 122 in one or more fillable forms, question/answer prompts, or other input interfaces based on the determined contextual information for each criteria in a clinical trial specification. Application 142 may accept or reject a response in relation to a criterion in a clinical trial specification based on the identified respondent in the contextual information associated with a criterion and the identity of the user of application 142. For example, if a criterion is identified by the second machine learning model as answerable by a physician and the user of application 142 is a type of user other than a physician, application 142 may reject the response. In another embodiment, clinical trial eligibility evaluations presented by application 142 may only present criteria that can be answered by the type of user using application 142 and save the user provided information to clinical trial data store 150 and/or patient data store 160 for further analysis. For some criteria that require patient consent or other patient-provided information, application 142 may block a user from providing information related to these criteria until the user indicates that the user has consulted with the patient or is currently consulting with the patient.

In some embodiments, application 142 may use temporal limitations in the contextual information associated with criteria in a clinical trial specification to determine whether to accept or reject a response to a request for information related to a particular criterion in the clinical trial specification. In some embodiments, application 142 may compare a time at which information related to a criteria is received by application 142 to temporal limitations in the contextual information to determine whether a provided response is valid. If the time at which information related to a criteria is received by application 142 is prior to a time identified by a temporal limitation in the contextual information determined by the second machine learning model, application 142 can reject the received information. The temporal limitations may indicate an absolute time or a gating event or set of events that must be performed on a patient prior to providing information about eligibility or disqualifying criteria to application 142.

While model trainer 130, application server 140, clinical trial data store 150, and patient data store 160 are illustrated as separate components in FIG. 1, it should be recognized that model trainer 130, application server 140, clinical trial data store 150, and patient data store 160 may be implemented on any number of computing systems, either as one or more standalone systems or in a distributed environment.

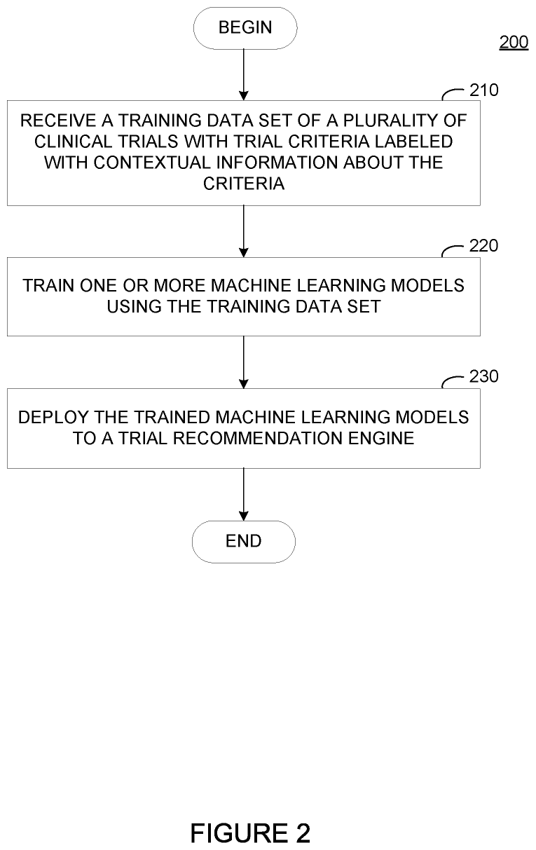

FIG. 2 illustrates example operations that may be performed by a machine learning model trainer to train a machine learning model for predicting intended respondents and timing for evaluating clinical trial eligibility for a patient, according to an embodiment. While FIG. 2 illustrates these operations as being performed in a single operation, it should be recognized by one of ordinary skill in the art that the operations illustrated in FIG. 2 may be executed separately to generate distinct machine learning models for identifying intended respondents for criteria in a clinical trial specification, intended times for response for these criteria, events that are to be performed prior to responding to criteria in a clinical trial specification, and the like.

Operations 200 begin at block 210, where a system (e.g., model trainer 130 illustrated in FIG. 1) receives a training data set of a plurality of clinical trials with trial criteria labeled with contextual information about the criteria. In some embodiments, the system may receive a first training data set with trial criteria labeled with the intended respondent for each criterion, a second training data set with trial criteria labeled with intended timing for response for each criterion, a third training data set with trial criteria labeled with events or other conditions that should be satisfied prior to responding to a criterion, and the like. Each of these training data sets may be used to train separate machine learning models that may be used by a trial recommendation system (e.g., trial recommendation engine 144 illustrated in FIG. 1) or an application (e.g., application 142 illustrated in FIG. 1) to filter clinical trial recommendations for a patient and/or aid a clinician or other user of application 142 in determining whether a patient is eligible to participate in a trial.

At block 220, the system trains one or more machine learning models using the training data set. The one or more machine learning models may be trained using supervised learning techniques. In some embodiments, the one or more machine learning models may comprise, for example, recurrent neural networks (RNNs) or other machine learning models that can use labeled training data to generate one or more algorithms for identifying contextual information for criteria in a clinical trial.

At block 230, the system deploys the trained machine learning models to a trial recommendation engine for use in identifying contextual information for criteria in a clinical trial, as discussed above.

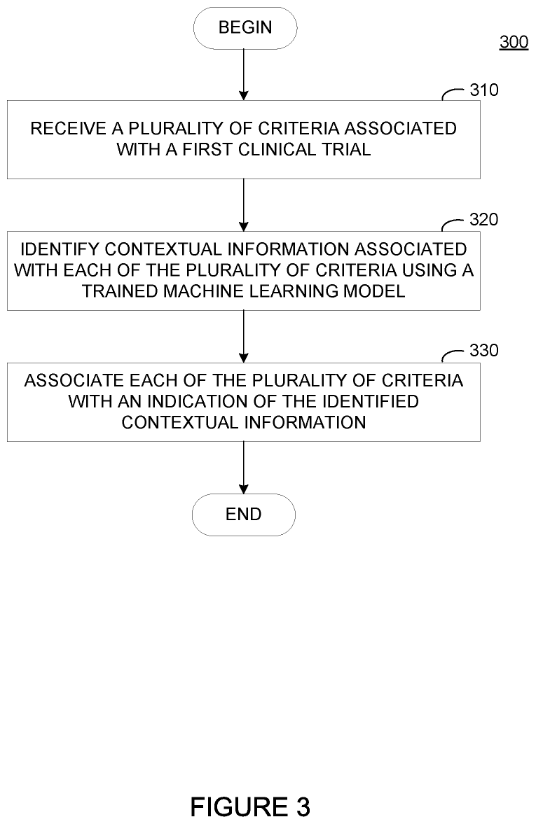

FIG. 3 illustrates example operations that may be performed by a system for predicting intended respondents and timing for evaluating clinical trial eligibility for a patient, according to an embodiment. While FIG. 3 illustrates these operations as being performed in a single operation, it should be recognized by one of ordinary skill in the art that the operations illustrated in FIG. 3 may be executed separately to identify intended respondents for criteria in a clinical trial specification, intended times for response for these criteria, events that are to be performed prior to responding to criteria in a clinical trial specification, and the like.

Operations 300 begin at block 310, where a system (e.g., application 142 illustrated in FIG. 1 and/or trial recommendation engine 144 illustrated in FIG. 1) receives a plurality of criteria associated with a first clinical trial. The plurality of criteria may be received from a specification for the first clinical trial and may include, for example, eligibility and disqualifying criteria for the trial. In some embodiments, the plurality of criteria may be augmented through the identification of implied criteria, which may be criteria that are not explicitly stated in the one or more documents but are determined to exist based on the inclusion of such criteria in clinically similar trials. Techniques for augmenting a clinical trial with implied criteria are described in U.S. patent application Ser. No. ______ of Will et al., filed ______, 2019 and entitled “Identifying Implied Criteria in Clinical Trials Using Machine Learning Techniques” (Attorney Docket No. P201805404US01), the contents of which are herein incorporated by reference.

At block 320, the system identifies contextual information associated with each of the plurality of criteria using a trained machine learning model. As discussed, the system can use a first machine learning model to identify intended respondents for each criterion specified for the first clinical trial. The intended respondents for a criterion may include, for example, a doctor, nurse, the patient, the patient's legal guardians, or other persons who may be qualified to provide information to resolve a trial criterion. A second machine learning model may be used to identify timing information for resolving a trial criterion. The timing information generally indicates when the intended user may provide an answer or other information to resolve a trial criterion. For example, the timing information may be defined relative to a procedure (e.g., a number of days/weeks/months after a procedure has been performed), as an absolute time (e.g., criteria that can be answered immediately), etc. A third machine learning model may be used to identify preconditions for resolving a trial criterion. These preconditions may specify that certain events should occur before the intended respondent provides information to resolve a trial criterion. The events may include, for example, performance of a particular medical procedure, performance of a specified type of scan within some amount of time prior to resolving the trial criterion, analysis of a tissue sample, or other events that may serve as a precondition to resolving the trial criterion.

At block 330, the system associates each of the plurality of criteria with an indication of the identified contextual information. As discussed above, the identified contextual information may be written to the trial specification for the first clinical trial to add contextual information to each criteria in the trial specification (including, in some embodiments, implied criteria identified from clinically similar trials). In embodiments where the machine learning models are deployed to application 142 to aid a user in determining whether a patient is eligible for participation in a clinical trial, the system may use the identified contextual information, for example, to control what criteria are visible to a given user, control whether a user can interact with each of the criteria defining the first clinical trial (e.g., provide information to resolve a trial criterion), control whether information provided by a user of application 142 may be accepted by application 142 to resolve a trial criterion, and the like. In embodiments where the machine learning models are deployed to trial recommendation engine 144, the identified contextual information may be used, along with patient medical history information from patient data store 150, to automatically resolve criteria for which information is available and determine, based on the patient medical history information, whether to display a clinical trial to a user of application 142 as a recommended clinical trial. Where a comparison of patient medical history information to clinical trial criteria indicates that the patient is not eligible for participation in a clinical trial, the clinical trial may be excluded from a recommended set of clinical trials for the patient. In contrast, where a comparison of patient medical history information to clinical trial criteria indicates that the patient is potentially eligible for participation in the clinical trial, the clinical trial may be included in the recommended set of clinical trials for the patient.

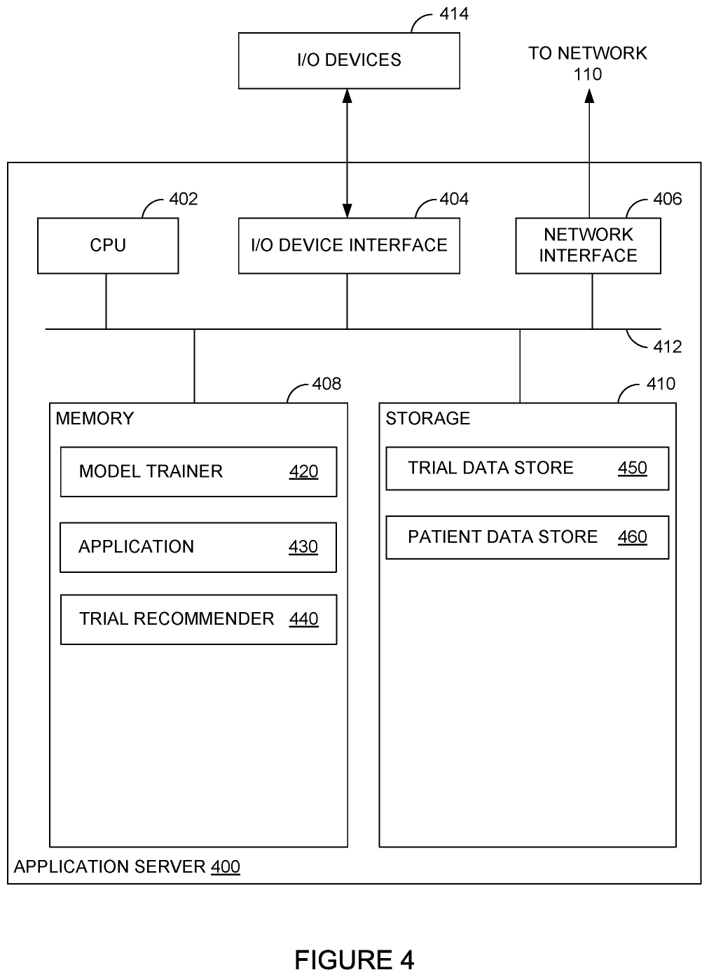

FIG. 4 illustrates an example application server 400 that uses machine learning techniques to identify contextual information about clinical trial criteria (e.g., intended respondents, timing of a response, preconditions for a response, etc.) and use the identified contextual information in a clinical trial management system, according to an embodiment. As shown, application server 400 includes, without limitation, a central processing unit 402, one or more I/O device interfaces 404, which may allow for the connection of various I/O devices 414 (e.g., keyboards, displays, mouse devices, pen input, etc.) to the entity analytics system 400, network interface 406, a memory 408, storage 410, and an interconnect 412.

CPU 402 may retrieve and execute programming instructions stored in the memory 408. Similarly, the CPU 402 may retrieve and store application residing in the memory 408. The interconnect 412 transmits programming instructions and application data among the CPU 402, I/O device interface 404, network interface 406, memory 408, and storage 410. CPU 402 is included to be representative of a single CPU, multiple CPUs, a single CPU having multiple processing cores, and the like. Additionally, the memory 408 is included to be representative of a random access memory. Furthermore, the storage 410 may be a disk drive. Although shown as a single unit, the storage 410 may be a combination of fixed and/or removable storage devices, such as fixed disc drives, solid state drives, removable memory cards or optical storage, network attached storage (NAS), or a storage area-network (SAN).

As illustrated, memory 408 includes a model trainer 420, an application 430, and a trial recommender 440. Model trainer 420 is generally configured to receive one or more training data sets of clinical trials for use in training one or more machine learning models. Each clinical trial in the training data sets may include a plurality of criteria, and each criterion may be labeled with contextual information identifying, for example, the intended respondent for the criterion, the intended time for responding to the criterion, preconditions for responding to the criterion, and the like. Model trainer 420 uses the received one or more training data sets to generate and deploy one or more machine learning models to application 430 and/or trial recommender 440 for use in identifying contextual information associated with a clinical trial. In some embodiments, the one or more machine learning models may be used to control how a user interacts with application 430 in evaluating patient eligibility for a clinical trial and/or whether clinical trials are included in a recommended set of clinical trials for a patient.

Application 430 may be configured to periodically or aperiodically analyze clinical trial specifications to identify contextual information for the criteria included in the clinical trial specifications and augment the clinical trial specifications with the identified contextual information. Generally, application 430 provides a clinical trial specification, including trial eligibility and disqualifying criteria, as input into the one or more machine learning models. The one or more machine learning models can identify contextual information (e.g., intended respondent, intended timing for response, preconditions for responding, etc.) for each criterion in the clinical trial specification and write the identified contextual information to the clinical trial specification for future use. In some embodiments, application 430 may present a selected, augmented, clinical trial specification to a user of application 430 and use the identified contextual information to control user interaction with the application 430 in determining patient eligibility for a trial, as discussed above.

Trial recommender 440 is generally configured to receive patient data as input and generate a set of recommended clinical trials for the patient for display to a user of application 430. The set of recommended clinical trials may be generated using a model that compares the received patient data to other clinically similar patients who have previously participated in clinical trials and identifies clinically similar trials to the trials that the clinically similar patients have participated in. In some embodiments, trial recommender 440 may use the one or more machine learning models to augment the set of recommended clinical trials with contextual information, and, based on the contextual information, determine whether a patient is ineligible for participation in a clinical trial using patient medical information and criteria that can be evaluated automatically (e.g., criteria that are not blocked for resolution by the non-occurrence of various preconditions). If trial recommender 440 determines that a patient is ineligible for participation in a clinical trial included in the set of recommended clinical trials, the clinical trial may be removed from the set of recommended clinical trials, thus reducing the number of false positives returned to a user of application 430 for further evaluation.

Storage 410, as illustrated, includes trial data store 450 and patient data store 460. Trial data store 450 generally represents a data repository in which details of previously performed and currently enrolling clinical trials are stored. Each trial stored in trial data store 450 generally includes eligibility and disqualifying criteria for the trial, operational characteristics of the trial, and the like. Patient data store 460 generally stores information about patients enrolled in previously performed trials and information about patients currently under consideration for inclusion in one or more clinical trials. As discussed, the patient information may be used to generate training data sets that are used to train machine learning models to recommend clinical trials for a patient.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

In the following, reference is made to embodiments presented in this disclosure. However, the scope of the present disclosure is not limited to specific described embodiments. Instead, any combination of the following features and elements, whether related to different embodiments or not, is contemplated to implement and practice contemplated embodiments. Furthermore, although embodiments disclosed herein may achieve advantages over other possible solutions or over the prior art, whether or not a particular advantage is achieved by a given embodiment is not limiting of the scope of the present disclosure. Thus, the following aspects, features, embodiments and advantages are merely illustrative and are not considered elements or limitations of the appended claims except where explicitly recited in a claim(s). Likewise, reference to “the invention” shall not be construed as a generalization of any inventive subject matter disclosed herein and shall not be considered to be an element or limitation of the appended claims except where explicitly recited in a claim(s).

Aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.”

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

While the foregoing is directed to embodiments of the present invention, other and further embodiments of the invention may be devised without departing from the basic scope thereof, and the scope thereof is determined by the claims that follow.

What is claimed is:

1. A method comprising:
training a machine learning (ML) model to identify an intended respondent for a criterion;
receiving a plurality of criteria associated with a first clinical trial;
determining a respective intended respondent for each of the plurality of criteria based on analyzing the plurality of criteria using the ML model; and
associating each of the plurality of criteria with an indication of the corresponding intended respondent.

2. The method of claim 1, wherein training the ML model comprises providing labeled training data to the ML model during a training phase.

3. The method of claim 1, wherein the intended respondent for each of the plurality of criteria indicates a qualification required to qualify a user to respond to the respective criterion.

4. The method of claim 3, the method further comprising:
receiving, from a first respondent, a first response to a first criterion of the plurality of criterion;
determining a qualification of the first respondent; and
upon determining that the qualification of the first respondent is below a qualification of the intended respondent for the first criterion, rejecting the first response.

5. The method of claim 1, the method further comprising:
training a second machine learning (ML) model to identify a time at which a criterion should be answered;
determining a respective time at which each of the plurality of criteria should be answered, based on analyzing the plurality of criteria using the second ML model; and
associating each of the plurality of criteria with an indication of the corresponding time.

6. The method of claim 5, the method further comprising:
receiving, at a first time, a first response to a first criterion of the plurality of criterion; and
upon determining that the first time is prior to the time at which the first criterion should be answered, rejecting the first response.

7. The method of claim 1, the method further comprising:
training a third machine learning (ML) model to identify a predefined event that must occur prior to answering a criterion;
determining a predefined event that must occur for each of the plurality of criteria, based on analyzing the plurality of criteria using the third ML model; and
associating each of the plurality of criteria with an indication of the corresponding predefined event.

8. The method of claim 5, the method further comprising:
receiving, at a first time, a first response to a first criterion of the plurality of criterion; and
upon determining that the predefined event corresponding to the first criterion has not occurred, rejecting the first response.

9. A system, comprising:
a processor; and
a memory having instructions stored thereon which, when executed by the processor, performs an operation, the operation comprising:
training a machine learning (ML) model to identify an intended respondent for a criterion;
receiving a plurality of criteria associated with a first clinical trial;
determining a respective intended respondent for each of the plurality of criteria based on analyzing the plurality of criteria using the ML model; and
associating each of the plurality of criteria with an indication of the corresponding intended respondent.

10. The system of claim 9, wherein training the ML model comprises providing labeled training data to the ML model during a training phase.

11. The system of claim 9, wherein the intended respondent for each of the plurality of criteria indicates a qualification required to qualify a user to respond to the respective criterion.

12. The system of claim 11, wherein the operation further comprises:
receiving, from a first respondent, a first response to a first criterion of the plurality of criterion;
determining a qualification of the first respondent; and
upon determining that the qualification of the first respondent is below a qualification of the intended respondent for the first criterion, rejecting the first response.

13. The system of claim 9, wherein the operation further comprises:
training a second machine learning (ML) model to identify a time at which a criterion should be answered;
determining a respective time at which each of the plurality of criteria should be answered, based on analyzing the plurality of criteria using the second ML model; and
associating each of the plurality of criteria with an indication of the corresponding time.

14. The system of claim 13, wherein the operation further comprises:
receiving, at a first time, a first response to a first criterion of the plurality of criterion; and
upon determining that the first time is prior to the time at which the first criterion should be answered, rejecting the first response.

15. The system of claim 9, wherein the operation further comprises:
training a third machine learning (ML) model to identify a predefined event that must occur prior to answering a criterion;
determining a predefined event that must occur for each of the plurality of criteria, based on analyzing the plurality of criteria using the third ML model; and
associating each of the plurality of criteria with an indication of the corresponding predefined event.

16. The system of claim 15, wherein the operation further comprises:
receiving, at a first time, a first response to a first criterion of the plurality of criterion; and
upon determining that the predefined event corresponding to the first criterion has not occurred, rejecting the first response.

17. A computer-readable medium having instructions stored thereon which, when executed by a processor, performs an operation, the operation comprising:
training a machine learning (ML) model to identify an intended respondent for a criterion;
receiving a plurality of criteria associated with a first clinical trial;
determining a respective intended respondent for each of the plurality of criteria based on analyzing the plurality of criteria using the ML model; and
associating each of the plurality of criteria with an indication of the corresponding intended respondent.

18. The computer-readable medium of claim 17, wherein the intended respondent for each of the plurality of criteria indicates a qualification required to qualify a user to respond to the respective criterion, and wherein the operation further comprises:
receiving, from a first respondent, a first response to a first criterion of the plurality of criterion;
determining a qualification of the first respondent; and
upon determining that the qualification of the first respondent is below a qualification of the intended respondent for the first criterion, rejecting the first response.

19. The computer-readable medium of claim 17, wherein the operation further comprises:
training a second machine learning (ML) model to identify a time at which a criterion should be answered;
determining a respective time at which each of the plurality of criteria should be answered, based on analyzing the plurality of criteria using the second ML model; and
associating each of the plurality of criteria with an indication of the corresponding time.

20. The computer-readable medium of claim 17, wherein the operation further comprises:
training a third machine learning (ML) model to identify a predefined event that must occur prior to answering a criterion;
determining a predefined event that must occur for each of the plurality of criteria, based on analyzing the plurality of criteria using the third ML model; and
associating each of the plurality of criteria with an indication of the corresponding predefined event.
